(19) United States
(12) Patent Application Publication
(10) Pub. No.: US 2016/0101030 A1
(43) Pub. Date: Apr. 14, 2016

(54) COSMETIC COMPOSITION FOR PREVENTING SKIN AGING

(71) Applicant: AMOREPACIFIC CORPORATION, Seoul (KR)

(72) Inventors: Hyeon Chung KIM, Yongin-si (KR); Hyun Hee KIM, Yongin-si (KR); Sung il PARK, Yongin-si (KR); Youn Joon KIM, Yongin-si (KR); Sang Hoon HAN, Yongin-si (KR)

(21) Appl. No.: 14/975,243

(22) Filed: Dec. 18, 2015

Related U.S. Application Data
(62) Division of application No. 13/807,665, filed on Dec. 28, 2012.

(30) Foreign Application Priority Data
Jun. 29, 2010 (KR) 10-2010-0061733
May 3, 2011 (KR) 10-2011-0042071

(51) Int. Cl. A61K 8/42; A61K 8/34; A61Q 17/04

(52) U.S. Cl. CPC A61K 8/42; A61Q 17/04; A61K 8/345; A61K 2800/592

(57) ABSTRACT

Provided is a composition including, as an active ingredient, at least one selected from the group consisting of amines, amides and polyols. The cosmetic composition has an effect of preventing aging and may be applied in various industrial fields, including cosmetics.

### CROSS-REFERENCE TO RELATED APPLICATION

This application is a divisional of U.S. patent application Ser. No. 13/807,665, filed on Dec. 28, 2012, which claims priority to PCT Application No. PCT/KR2011/004546, filed on Jun. 22, 2011, which claims priority to Korean Patent Application No. 10-2010-0061733, filed on Jun. 29, 2010, and Korean Patent Application No. 10-2011-0042071, filed on May 3, 2011, and all the benefits accruing therefrom under 35 U.S.C. §119, the contents of which in its entirety are herein incorporated by reference.

### TECHNICAL FIELD

This disclosure relates to a cosmetic composition for preventing aging caused by infrared (IR) rays or the like.

### BACKGROUND ART

It is generally known that ultraviolet (UV) rays having a shorter wavelength than visible rays may adversely affect human skin. Thus, many studies have been conducted to develop UV-blocking products.

However, effects of infrared (IR) rays upon skin are not particularly known to date. IR rays occupy 80% of the sunlight, have a lower tendency to reflect or scatter by microparticles in the atmosphere than UV or visible rays, and transmit through the air to reach the ground surface while not being interrupted by oxygen or nitrogen molecules in the air.

Recently, it has been demonstrated that IR rays having a blood circulation-stimulating effect and hyperthermia effect may adversely affect human skin. Particularly, it has been shown that IR rays may stimulate formation of skin wrinkles. The mechanism of skin wrinkle formation caused by IR rays is different from the mechanism of skin wrinkle formation caused by UV rays. Therefore, application of known UV blocking agents onto skin when the skin is exposed to sunlight may not prevent skin aging caused by IR rays.

Therefore, there is a need for developing a formulation for blocking rays over a wide range of wavelengths, the formulation being capable of blocking IR rays as well as UV rays.

### DISCLOSURE

### Technical Problem

This disclosure is directed to providing a cosmetic composition for preventing aging caused by infrared (IR) rays etc., when applied onto skin.

### Technical Solution

In one general aspect, there is provided a cosmetic composition including, as an active ingredient, at least one selected from the group consisting of amines, amides and polyols.

### Advantageous Effects

The cosmetic composition according to an embodiment of this disclosure may prevent aging by blocking ultraviolet (UV) rays and infrared (IR) rays, and thus may be used widely in the field of beauty and cosmetics.

### DESCRIPTION OF DRAWINGS

The above and other aspects, features and advantages of the disclosed exemplary embodiments will be more apparent from the following detailed description taken in conjunction with the accompanying drawings in which:

FIGS. 1 to 4 are graphs showing the results of infrared protectabilities measured by Fourier-transform infrared spectroscopy; and

FIG. 5 is a graph showing the results of near-IR protectabilities measured by Fourier-transform near infrared spectroscopy.

### BEST MODE

Exemplary embodiments will be described more fully hereinafter. This disclosure may, however, be embodied in many different forms and should not be construed as limited to the exemplary embodiments set forth therein. Rather, these exemplary embodiments are provided so that this disclosure will be thorough and complete, and will fully convey the scope of this disclosure to those skilled in the art. In the description, details of well-known features and techniques may be omitted to avoid unnecessarily obscuring the presented embodiments.

In one aspect, the composition according to an embodiment of this disclosure is a cosmetic composition for preventing and improving aging, and includes at least one selected from the group consisting of amines, amides and polyols, as an active ingredient.

In one embodiment, the cosmetic composition is a composition for preventing thermal aging, and more particularly, for blocking heat, or near-infrared (IR) and IR rays. The cosmetic composition may further include an ultraviolet (UV) blocking agent, and prevent thermal aging and photo-aging at the same time. For example, the composition according to this embodiment blocks UV A, UV B, near-IR and IR rays at the same time.

As used herein, the term ‘thermal aging’ means a phenomena of skin aging caused by heat, and includes skin aging that proceeds when the skin is exposed continuously to a high temperature, as well as skin aging that proceeds when the skin is exposed to IR rays. Thermal aging may be found frequently in those working in places requiring long-term exposure to a high temperature, or staying in Korean dry sauna rooms or sauna baths for a long time. Thermal skin aging may also proceed when the skin is exposed to IR rays of the sunlight. Particularly, IR and near-IR rays have been thought heretofore to have no harmful effect upon skin. However, repeated exposure to IR and near-IR rays causes a rapid drop in synthesis of collagen and an increase in synthesis of matrix metalloproteinase (MMP), which is a collagen-decomposing enzyme, resulting in skin aging.

As used herein, the term ‘photo-aging’ means skin aging occurring upon the exposure to sunlight, particularly, skin aging caused by the exposure to UV rays. Typical examples of phenomena occurring when photo-aging proceeds include thick and deep wrinkles on the exposed portions, such as faces, necks, arms and hands, and pigment deposition, such as age spots and blemishes. Unlike biological aging including simple skin thinning and loosening, photo-aged skin has materials similar to abnormal elastic fibers packed closely in the upper derma. Thus, the derma may not perform their original function of supporting skin and retaining moisture. UV rays cause direct gene damages in skin cells, leading to skin aging, skin cancers and dermatitis, thereby causing generation of a large amount of active oxygen species secondarily and further aging by oxidation. As a result, this inhibits formation of collagenous and elastic fibers forming the derma and stimulates functions of collagen-decomposing enzymes, leading to a drop in collagenous and elastic fibers.

UV rays mean electromagnetic waves having a shorter wavelength than visible rays, i.e. a wavelength of 397-10 nm, when dispersing the light emitted by the sun through a prism. IR rays are referred to as heat rays while UV rays are also referred to as actinic rays due to their strong chemical actions. In addition, UV rays may be further classified, depending on the magnitude of wavelength, into near-UV (290 nm or more), crystal-range UV (290-190 nm, a wavelength range transmitted through crystal), Schumann rays (190-120 nm), Reimann rays (120-60 nm) and Millikan rays (60 nm or less). UV rays having a wavelength of 190 nm or less may also be referred to as far-UV rays.

IR rays mean electromagnetic waves present at the external portion of the end of the red line, when dispersing the light emitted by the sun through a prism. IR rays are characterized by their stronger thermal action than visible or UV rays. Thus, IR rays may also be referred to as heat rays. IR rays may be classified, depending on the magnitude of wavelength, into near-IR (780-3000 nm), IR (3000-25000 nm) and far-IR (25000 nm or more).

It has been found that the cosmetic composition disclosed herein blocks IR rays (including near-IR and IR rays), which, otherwise, may not be blocked by known UV blocking agents.

In one embodiment, a cosmetic composition including at least one selected from the group consisting of amines, amides and polyols provides an excellent effect of blocking near-IR and IR rays, as demonstrated by the following test examples.

As used herein, the term ‘amines’ is a generic term of materials having an amine group (—NH, —NH₂) as a functional group. In one embodiment, amines may include urea, or a salt or derivative thereof. Urea is an organic compound represented by the following Chemical Formula 1 (CO(NH₂)₂), is a colorless crystalline material, and is a final decomposition product of protein metabolism.

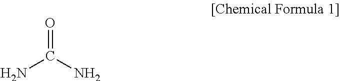

In another embodiment, amines may include at least one selected from the group consisting of acetyl glucosamine, polyglutamic acid, 2-pyrrolidone-5-carboxylic acid (PCA), alanine, lysine and glycine, or a salt or derivative thereof.

According to an embodiment, amines may be present in an amount of 0.001-20 wt %, more particularly 1-10 wt %, based on the total weight of the composition. When amines are present in an excessively small amount, the composition provides poor quality as a near-IR and IR absorbing agent, so that it may show an insufficient effect of preventing thermal aging. On the other hand, when amines are present in an excessively large amount, the composition may cause skin irritation or may provide a bad touch in use.

As used herein, the term ‘amides’ is a generic term of materials having an amide group (—CONH₂) as a functional group. In one embodiment, amides may include niacinamide, or a salt or derivative thereof. Niacinamide has a structural formula represented by the following Chemical Formula 2, and is one of vitamin B complexes.

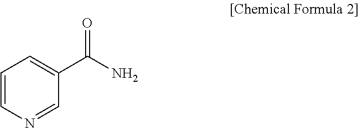

In another embodiment, amides may include at least one selected from the group consisting of pantethine and panthenol, or a salt or derivative thereof.

According to an embodiment, amides may be present in an amount of 0.001-20 wt %, more particularly 1-10 wt %, based on the total weight of the composition. When amides are present in an excessively small amount, the composition provides poor quality as a near-IR and IR absorbing agent, so that it may show an insufficient effect of preventing thermal aging. On the other hand, when amides are present in an excessively large amount, the composition may cause skin irritation or may provide a bad touch in use.

As used herein, the term ‘polyols’ is a generic term of materials having multiple hydroxyl groups (—OH) as a functional group. In one embodiment, polyols may include inositol, or a salt or derivative thereof. Inositol has a structural formula represented by the following Chemical Formula 3, and is an alicyclic hexahydric alcohol having a structure of cyclohexane-1,2,3,4,5,6-hexanol.

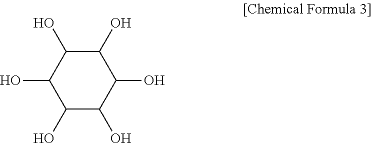 [SMILES: OC1C(O)C(O)C(O)C(O)C1O]

In another embodiment, polyols may include at least one selected from the group consisting of erythritol, glycerin, maltitol, mannitol, sorbitol, xylitol, butylenes glycol, pentylene glycol and propylene glycol, or a salt or derivative thereof.

According to an embodiment, polyols may be present in an amount of 0.001 wt % or higher, particularly 0.001-99.9 wt %, and more particularly 1-10 wt %, based on the total weight of the composition. When polyols are present in an excessively small amount, the composition provides poor quality as a near-IR and IR absorbing agent, so that it may show an insufficient effect of preventing thermal aging. On the other hand, when amides are present in an excessively large amount, the composition may cause skin irritation or may provide a bad touch in use.

The cosmetic composition disclosed herein prevents thermal aging of skin. Particularly, it is shown that the cosmetic composition prevents or blocks progress of aging caused by the exposure of skin to heat, IR and/or near-IR rays. In one embodiment, the cosmetic composition may further include a UV-blocking agent. According to an embodiment, the cosmetic composition further including a UV-blocking agent prevents thermal aging caused by IR absorption and photo-aging caused by UV absorption at the same time. There is no particular limitation in the UV-blocking agent, as long as it has an effect of blocking UV rays. Such UV-blocking agents may include organic or inorganic UV-blocking agents.

Particular examples of organic UV-blocking agents may include at least one selected from the group consisting of octocrylene, octyl dimethyl para-aminobenzoic acid (PABA), octyl methoxycinnamate, octyl salicylate, octyl triazone, butylmethoxy dibenzoylmethane, benzopheneone, oxybenzone, sulisobenzone, dioxybenzone, Mexoryl-SX, ensulizole, meradimate, avobenzone, isoamyl paramethoxycinnamate, bis-ethylhexyloxyphenol methoxyphenyltriazine and methylene bis-benzotriazolyl tetramethylbutylphenol. Particular examples of inorganic UV-blocking agents may include at least one selected from the group consisting of titanium dioxide, titanium oxide, zinc oxide, boron nitride, cerium oxide and iron oxides. Such organic or inorganic UV-blocking agents may be used with no particular limitation, as long as they do not adversely affect the effect of preventing thermal aging when added to the cosmetic composition disclosed herein.

The cosmetic composition disclosed herein may be formulated in various manners with no particular limitation. For example, the cosmetic composition may be formulated into astringent, nutrient lotion, nutrient cream, massage cream, pack, sun cream, foundation or makeup base. In one embodiment, the cosmetic composition may be one blocking near-IR and IR rays. In another embodiment, the cosmetic composition may be one blocking UV rays and IR rays at the same time. In still another embodiment, the cosmetic composition may be one blocking UV A (320-400 nm), UV B (280-320 nm), near-IR and IR rays at the same time. Each type of formulation may be provided easily in a manner known to those skilled in the art by selecting various ingredients other than the essential ingredients depending on the intended use, etc.

### MODE FOR INVENTION

The examples and experiments will now be described. The following examples and experiments are for illustrative purposes only and not intended to limit the scope of this disclosure.

### EXAMPLES 1 TO 11 AND COMPARATIVE EXAMPLE

The cosmetic compositions of Examples 1 to 11 and Comparative Example are provided by using the ingredients as shown in the following Tables 1 and 2.

TABLE 1

| Ingredients | Comp. Ex. | Ex. 1 | Ex. 2 | Ex. 3 | Ex. 4 | Ex. 5 |
|---|---|---|---|---|---|---|
| Deionized | to | to | to | to | to | to |
| water | 100 | 100 | 100 | 100 | 100 | 100 |
| EDTA-2Na | q.s. | q.s. | q.s. | q.s. | q.s. | q.s. |
| Inositol | — | 5 | — | — | — | — |
| Urea | — | — | 5 | — | — | — |
| Niacinamide | — | — | — | 5 | — | — |
| N-acetyl | — | — | — | — | 5 | — |
| glucosamine | | | | | | |
| Polyglutamic | — | — | — | — | — | 5 |
| acid | | | | | | |
| Phenoxyethanol | q.s. | q.s. | q.s. | q.s. | q.s. | q.s. |
| Cetearyl alcohol | 1 | 1 | 1 | 1 | 1 | 1 |
| Phytosqualane | 5 | 5 | 5 | 5 | 5 | 5 |
| Cetearyl | 1 | 1 | 1 | 1 | 1 | 1 |
| alcohol/ | | | | | | |
| cetearyl | | | | | | |
| glucoside | | | | | | |
| mixing ratio | | | | | | |
| Tromethamine | q.s. | q.s. | q.s. | q.s. | q.s. | q.s. |
| Carbomer | q.s. | q.s. | q.s. | q.s. | q.s. | q.s. |

TABLE 2

| Ingredients | Ex. 6 | Ex. 7 | Ex. 8 | Ex. 9 | Ex. 10 | Ex. 11 |
|---|---|---|---|---|---|---|
| Deionized | to | to | to | to | to | to |
| water | 100 | 100 | 100 | 100 | 100 | 100 |
| EDTA-2Na | q.s. | q.s. | q.s. | q.s. | q.s. | q.s. |
| L-alanine | 5 | — | — | — | — | — |
| L-lysine HCl | — | 5 | — | — | — | — |
| Panthenol | — | — | 5 | — | — | — |
| Erythritol | — | — | — | 5 | — | — |
| Mannitol | — | — | — | — | 5 | — |
| Xylitol | | | | | | 5 |
| Phenoxyethanol | q.s. | q.s. | q.s. | q.s. | q.s. | q.s. |
| Cetearyl alcohol | 1 | 1 | 1 | 1 | 1 | 1 |
| Phytosqualane | 5 | 5 | 5 | 5 | 5 | 5 |
| Cetearyl | 1 | 1 | 1 | 1 | 1 | 1 |
| alcohol/ | | | | | | |
| cetearyl | | | | | | |
| glucoside | | | | | | |
| mixing ratio | | | | | | |
| Tromethamine | q.s. | q.s. | q.s. | q.s. | q.s. | q.s. |
| Carbomer | q.s. | q.s. | q.s. | q.s. | q.s. | q.s. |

### TEST EXAMPLE 1

### Determination of IR Blocking Capability

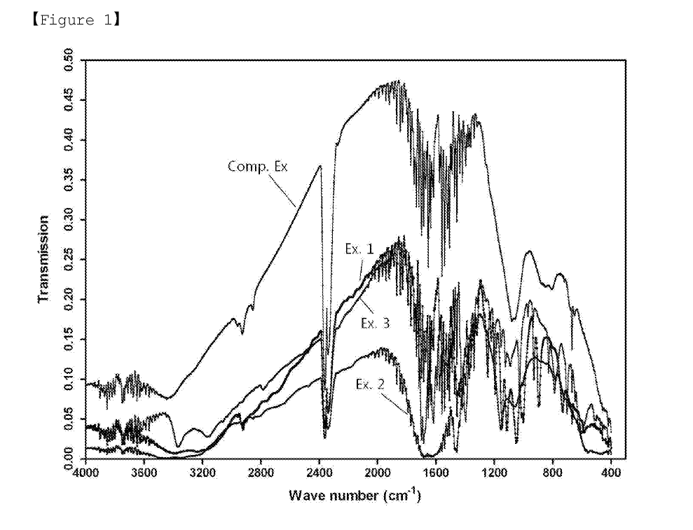

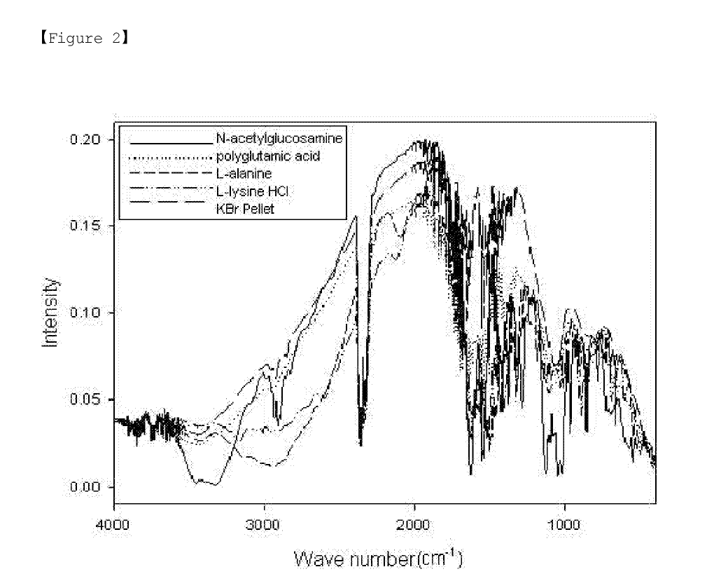

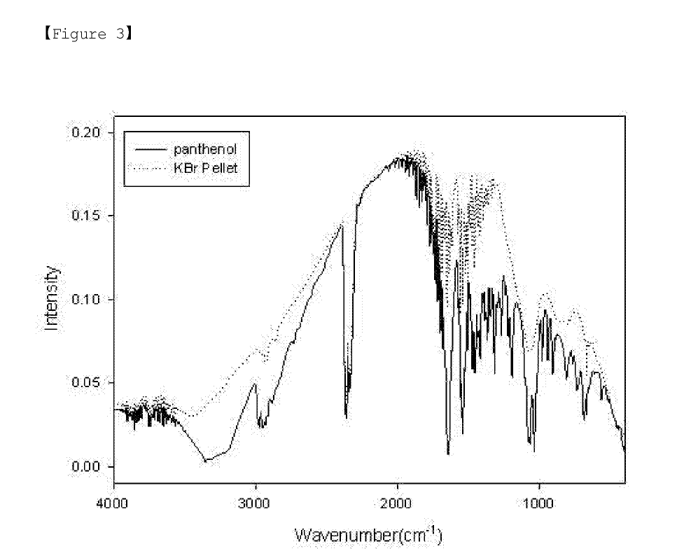

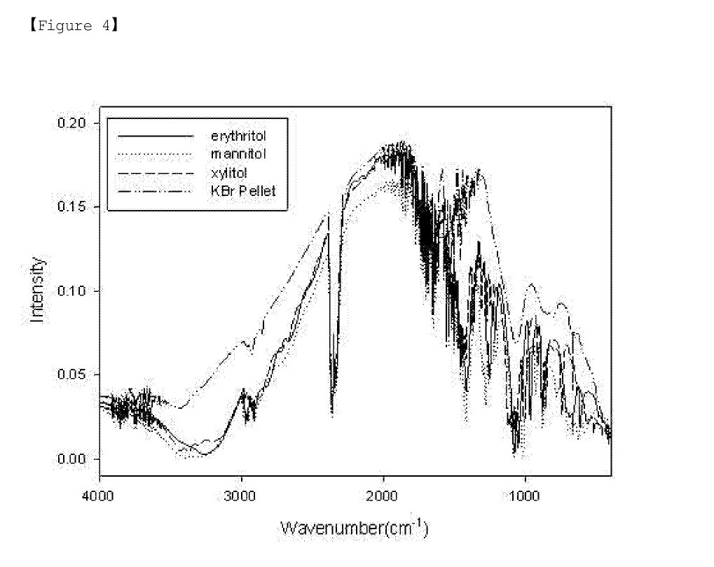

The infrared (IR) protectabilities of the compositions according to Examples 1 to 11 and Comparative Example are determined. Particularly, IR protectabilities are determined through a Fourier Transform Infrared Spectrometer, Tensor 27 (available from Bruker Co.). Samples to be analyzed are provided in the form of pellets and the test is carried out by measuring the transmission of each sample. The test results are shown in FIGS. 1 to 4, wherein the wavelength range shown at the x-axis is expressed in the unit of wave number (cm⁻¹).

Referring to FIGS. 1 to 4, it is said that a lower transmission in the IR range means higher IR blocking capability. It can be seen that the cosmetic compositions of Examples 1 to 11 have higher IR protectabilities, as compared to the composition of Comparative Example.

### TEST EXAMPLE 2

### Determination of Near-IR Absorbance

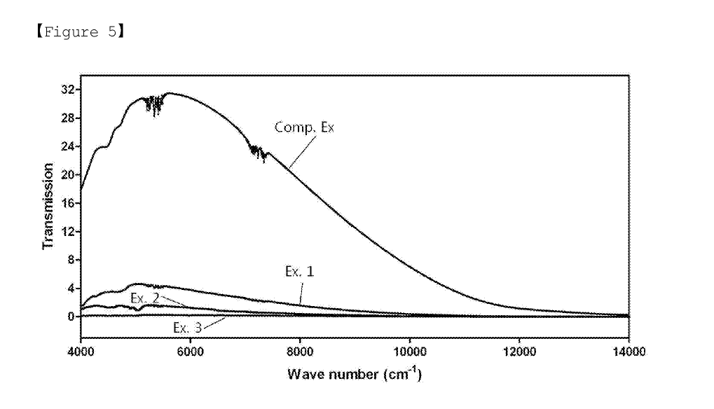

The near-IR protectabilities of the compositions according to Examples 1 to 3 and Comparative Example are determined. Particularly, near-IR protectabilities are determined through a Fourier Transform Infrared Spectrometer, Tensor 27 (available from Bruker Co.). Samples to be analyzed are provided in the form of pellets and the test is carried out by measuring the transmission of each sample. The test results are shown in FIG. 5, wherein the wavelength range shown at the x-axis is expressed in the unit of wave number (cm⁻¹).

Referring to FIG. 5, it is said that a lower transmission at the near-IR range means higher near-IR blocking capability. It can be seen that the cosmetic compositions of Examples 1 to 3 have higher near-IR protectabilities, as compared to the composition of Comparative Example. Particularly, the cosmetic compositions according to Examples 2 and 3 block most of near-IR rays in a region of 4,000-10,000 cm⁻¹.

The formulation examples will now be described. However, the cosmetic composition disclosed herein may be formulated in various forms other than those described hereinafter. The following formulation examples are for illustrative purposes only and not intended to limit the scope of this disclosure.

### FORMULATION EXAMPLE 1

### Lotion

unit: wt %

| | |
|---|---|
| Inositol (Ex. 3) | 3.00 |
| L-ascorbic acid 2-phosphate magnesium salt | 1.00 |
| Water soluble collagen (1% aq. solution) | 1.00 |
| Sodium citrate | 0.10 |
| Citric acid | 0.05 |
| Licorice extract | 0.20 |
| 1,3-Butylene glycol | 3.00 |
| Deionized water | Balance |

### FORMULATION EXAMPLE 2

### Cream

unit: wt %

| | |
|---|---|
| Niacinamide (Ex. 2) | 1.00 |
| Polyethylene glycol monostearate | 2.00 |
| Self-emulsified glycerin monostearate | 5.00 |
| Cetyl alcohol | 4.00 |
| Squalene | 6.00 |
| Tri-2-ethylhexanoic acid glyceryl ester | 6.00 |
| Spingoglycolipid | 1.00 |
| 1,3-Butylene glycol | 7.00 |
| Deionized water | Balance |

### FORMULATION EXAMPLE 3

### Pack

unit: wt %

| | |
|---|---|
| Urea (Ex. 1) | 5.00 |
| Polyvinyl alcohol | 13.00 |
| L-ascorbic acid 2-phosphate magnesium salt | 1.00 |
| Lauroylhydroxyproline | 1.00 |
| Water soluble collagen (1% aq. solution) | 2.00 |
| 1,3-Butylene glycol | 3.00 |
| Ethanol | 5.00 |
| Deionized water | Balance |

### FORMULATION EXAMPLE 4

### Essence

unit: wt %

| | |
|---|---|
| Inositol (Ex. 3) | 2.00 |
| Hydroxyethylene cellulose (2% aq. solution) | 12.00 |
| Xanthan gum (2% aq. solution) | 2.00 |
| 1,3-Butylene glycol | 6.00 |
| Glycerin | 4.00 |
| Sodium hyaluronate (1% aq. solution) | 5.00 |
| Deionized water | Balance |

While the exemplary embodiments have been shown and described, it will be understood by those skilled in the art that various changes in form and details may be made thereto without departing from the spirit and scope of this disclosure as defined by the appended claims.

In addition, many modifications can be made to adapt a particular situation or material to the teachings of this disclosure without departing from the essential scope thereof. Therefore, it is intended that this disclosure not be limited to the particular exemplary embodiments disclosed as the best mode contemplated for carrying out this disclosure, but that this disclosure will include all embodiments falling within the scope of the appended claims.

1. A method for blocking near-infrared (IR) and IR rays, comprising administering at least one selected from the group consisting of amines and polyols to a subject in need thereof.

2. The method according to claim 1, wherein the at least one selected from the group consisting of amines and polyols prevent thermal aging.

3. The method according to claim 1, wherein the at least one selected from the group consisting of amines and polyols block heat.

4. The method according to claim 1, further comprising administering an ultraviolet (UV)-blocking agent.

5. The method according to claim 4, wherein the at least one selected from the group consisting of amines and polyols, and an ultraviolet (UV)-blocking agent prevent thermal aging and photo-aging simultaneously.

6. The method according to claim 4, wherein the at least one selected from the group consisting of amines and polyols, and an ultraviolet (UV)-blocking agent block UV A, UV B, near-IR and IR rays simultaneously.

7. The method according to claim 1, wherein the at least one selected from the group consisting of amines and polyols are administered in the form of a cosmetic composition, and the cosmetic composition comprise the amines in an amount of 0.001-20 wt % based on the total weight of the composition.

8. The method according to claim 1, wherein the amines are at least one selected from the group consisting of urea, acetyl glucosamine, polyglutamic acid, 2-pyrrolidone-5-carboxylic acid (PCA), alanine, lysine, glycine, salts thereof and derivatives thereof.

9. The method according to claim 1, wherein the at least one selected from the group consisting of amines and polyols are administered in the form of a cosmetic composition, and the cosmetic composition comprises the polyols in an amount of 0. 001-99.9 wt % based on the total weight of the composition.

10. The method according to claim 1, wherein the polyols are at least one selected from the group consisting of inositol, erythritol, glycerin, maltitol, mannitol, sorbitol, xylitol, butylenes glycol, pentylene glycol, propylene glycol, salts thereof and derivatives thereof.
